(19) United States
(12) Patent Application Publication
(10) Pub. No.: US 2024/0368055 A1
(43) Pub. Date: Nov. 7, 2024

(54) PROCESS FOR THE SEPARATION OF OLEFINS AND LOW MOLECULAR WEIGHT HYDROCARBONS IN METHANOL TO OLEFIN PROCESSES

(71) Applicant: Braskem S.A., Camaçari (BR)

(72) Inventors: Nayana Pereira Andrade, São Paulo City (BR); Márcio das Virgens Rebouças, São Paulo City (BR); Márcio Henrique dos Santos Andrade, São Paulo City (BR); Karoline Dantas Brito, São Paulo City (BR); Romildo Pereira Brito, São Paulo City (BR)

(73) Assignee: Braskem S.A., Camaçari (BR)

(21) Appl. No.: 18/653,775

(22) Filed: May 2, 2024

Related U.S. Application Data
(60) Provisional application No. 63/627,320, filed on Jan. 31, 2024.
(60) Provisional application No. 63/463,388, filed on May 2, 2023.

(51) Int. Cl. C07C 1/24; C07C 7/04; C07C 7/09

(52) U.S. Cl. CPC C07C 1/24; C07C 7/04; C07C 7/09

(57) ABSTRACT

A process of purifying downstream methanol-to-olefin streams comprising separating a downstream methanol-to-olefin stream comprising one or more light and heavy components in a first column into a light component stream and a heavy component stream, purifying a light component stream with a first dividing wall distillation column thereby producing an ethylene fraction and distilling the heavy component stream thereby forming a propylene fraction.

### BACKGROUND

The demand for olefins such as ethylene and propylene is ever increasing and is currently heavily reliant on the production of these olefins through the steam cracking of naphtha. Therefore, alternative processes are being explored in order to more sustainably meet the needs of the future. The recent increase in biogas production and discovery of new natural gas reserves have provided an opportunity to explore obtaining olefins using methanol. These processes have been coined as the methanol-to-olefin (MTO) processes and have proved to be a potentially sustainable alternative to traditional olefin production processes.

Since the introduction of the MTO process, the technology has evolved to allow for the precise control over the ratio of olefins produced making the ever-changing market demands in volume of the olefins easy to attain. The process involves 4 stages: 1) gas reform, 2) conversion of synthesis gas into methanol, 3) methanol-to-olefin process and 4) olefin separation. The olefin separation stage typically consists of a sequence of distillation columns in order to separate propane/propylene and ethane/ethylene and as such, is energetically expensive due to the volatility of the components. In addition, conventional distillation processes have low energy efficiency, implying high energy consumption in the product recovery stage. Therefore, there remains a need to find a process to produce olefins from MTO where the separation of the olefins is more energetically feasible.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, embodiments disclosed herein relate to a process of purifying downstream methanol-to-olefin streams that includes separating a downstream methanol-to-olefin stream including one or more light and heavy components in a first column into a light component stream and a heavy component stream, purifying a light component stream with a first dividing wall distillation column thereby producing an ethylene fraction and distilling the heavy component stream thereby forming a propylene fraction.

In another aspect, embodiments herein relate to a system for purifying downstream methanol-to-olefin streams including a first column for separating the downstream methanol-to-olefin stream a light component stream and a heavy component stream, a first dividing wall distillation column fluidly connected to the first column, receiving the light component stream and producing an ethylene fraction therefrom, and a second column fluidly connected to and receiving the heavy component stream from the first column, the second column configured to isolate a propylene fraction from the heavy component stream.

Other aspects and advantages of the claimed subject matter will be apparent from the following description and the appended claims.

### BRIEF DESCRIPTION OF DRAWINGS

FIG. 1 depicts a dividing wall column according to one or more embodiments.

FIG. 2 shows a process flow diagram according to one or more embodiments.

FIG. 3 shows a process flow diagram according to at least one embodiment.

FIG. 4 shows a UOP/HYDRO MTO process stream recovery section including six distillation columns used in Comparative Example 1.

FIG. 5 shows a configuration used in Example 2.

FIGS. 6A-6F present the results of the influence of the three manipulated variables (RR1, FL1 and FV2) on the thermal load of the DWC-1 distillation column reboiler and on ethylene purity.

FIG. 7 shows a configuration used in Example 3.

FIGS. 8A-8F show the results for the sensitivity analysis performed to determine the reflux ratio (RR2) and removals of liquid (FL3) and vapor (FV4) from the main column (DWC-2).

FIG. 9 presents energy costs for the three configurations.

FIG. 10 presents the economic results for the three configurations.

FIG. 11 shows an alternative design for the synthesis to methanol section described in Example 4.

FIG. 12 shows an alternative design for the methanol to olefin reaction section described in Example 4.

FIG. 13 shows a configuration used in Example 4.

FIG. 14 shows a comparison of energy removed in the three stages of the MTO process between the base design and the alternative design.

FIG. 15 shows a comparison of energy supply in the three stages of the MTO process between the base design and the alternative design.

### DETAILED DESCRIPTION

Embodiments disclosed herein are directed to the use of dividing wall distillation columns for the downstream separation of streams from methanol-to-olefin (MTO) processes. Olefins typically display close-to-unity relative volatilities which makes them difficult to separate from one another in production processes. However, the use of dividing wall columns (DWCs) allows for lower energy consumption compared to traditional processes. The integration of the DWCs downstream of the MTO process allows for control of the olefin fractions and their resulting purity. In order to overcome the technical barriers discussed above, the present disclosure provides methods for purifying downstream MTO streams using a series of distillation columns, including dividing wall columns.

Use of one or more dividing wall columns may advantageously allow for not only the separation of downstream MTO process but also better control of the output. In addition, the shared reboiler and condenser of the dividing wall columns allow for considerable energetic and cost savings associated with the separations. Hence, including one or more DWCs in the downstream MTO process favors increased ratios of output for the olefins as well as product purity.

Embodiments disclosed herein relate to a system and process of purifying downstream MTO streams including one or more dividing wall columns, where the MTO stream is separated into individual fractions of hydrocarbon components. The purification process includes a mixture of conventional distillation and dividing wall columns.

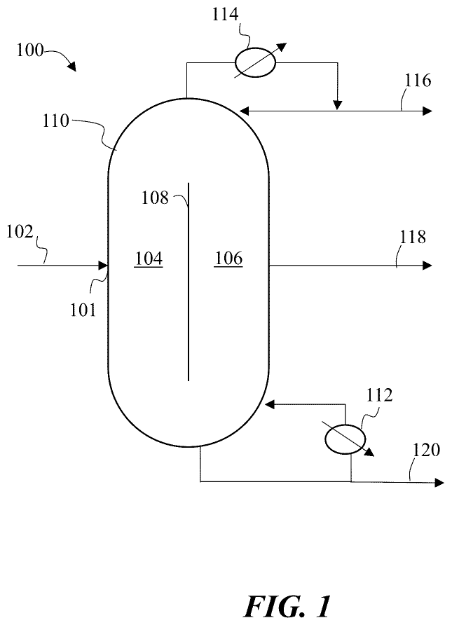

In the present disclosure, DWCs are used in addition to conventional distillation columns. A typical dividing wall column for use in the process according to one or more embodiments is shown in FIG. 1. The DWC 100 comprises an inlet 101 for a feed stream 102, as well as a main column 104 and a post-fractionation section 106. The main column 104 and post-fractionation sections 106 are separated from one another by a partition wall 108 that runs parallel and lengthwise with the column body 110 to divide column 100 into the two sections. The DWC 100 further comprises a single reboiler 112 and condenser 114. The lower boiling point components of the stream are vaporized and condensed then subsequently removed as a first component 116 or top product, medium boilers are removed as a second component 118 or side product and high boilers are removed as third component 120 or bottom product. In operation, when feed stream 102 is fed into DWC 100, it feeds into main column 104. First component 116 and a portion of second component 118 will go over partition wall 108 to upper rectification section, while third component 120 and the remaining portion of second component 118 will go under the wall to the lower stripping section.

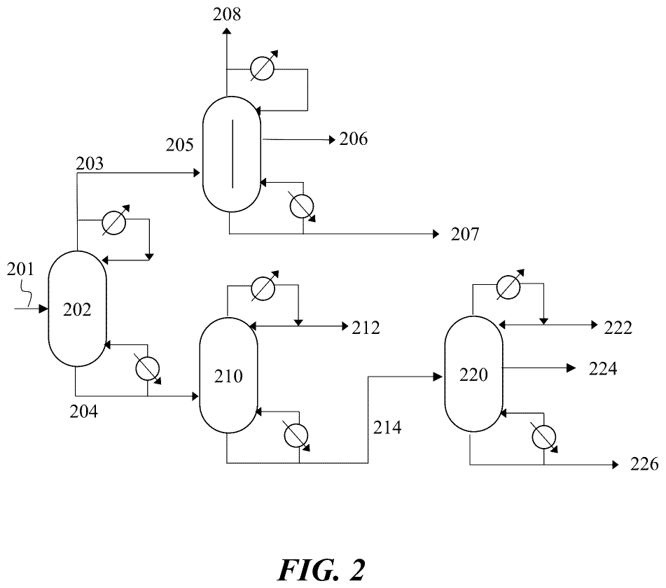

Referring to FIG. 2, a generalized process flow diagram is shown to represent the process for the purification of downstream MTO streams 201 according to one or more embodiments. First, the downstream MTO stream feeds a first column 202 that separates the stream into a light component stream 203 and a heavy component stream 204. The light component stream 203 from the first column 202 feeds the first dividing wall column 205 which separates ethylene 206 from ethane 207 and tail gases 208. The heavy component stream 204 from the first column 202 feeds the second column 210 where a propylene fraction 212 is separated. The heavy component 214 from the second column 210 feeds a third column 220 which separates the heavy component stream 214 into fractions. It is envisioned that the design of any columns used in the present embodiments involves considerations of parameters such as product compositions, product flowrates, operating pressure, total number of trays, feed tray location, reflux ratio, reboiler heat input, condenser heat removal, column diameter, and column height. A person of ordinary skill in the art would understand that the parameters of any distillation column rely on input conditions (composition of the imputed load and operational temperatures and pressures), output conditions (desired product composition and operational temperatures and pressures).

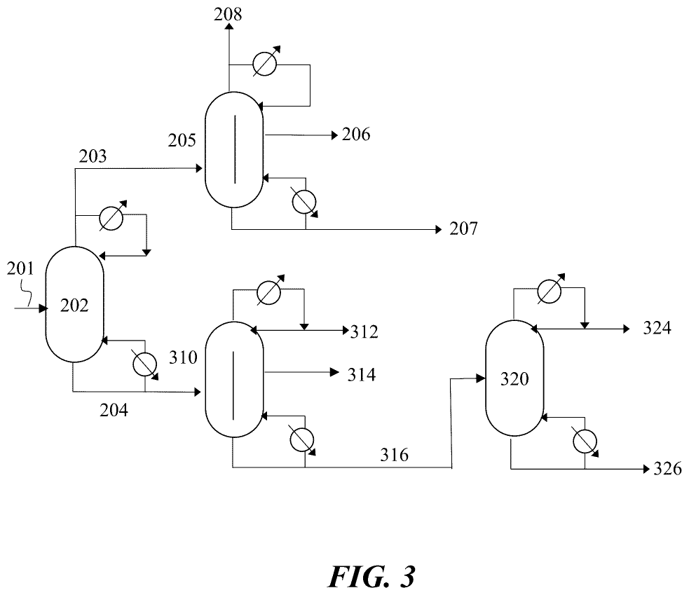

Alternative to the second column 210, the heavy component stream 204 from the first column 202 may feed a second dividing wall column 310, as shown in FIG. 3. The second dividing wall column 310 separates the propylene fraction 312 and isolates propane 314 from a heavy C4/C5 component stream 316. The heavy C4/C5 component stream 316 feeds a third column 320. The third column 320 separates the heavy C4/C5 component stream 316 into fractions.

The above described DWCs may be used to purify downstream MTO streams. According to one or more embodiments, the downstream methanol-to-olefin streams 201 that feed the first column 202 may comprise components selected from the group consisting of methane, ethane, propane, nitrogen, hydrogen, carbon monoxide, ethylene, propylene, mixed-C4, mixed-C5 and mixtures thereof.

As referred to above, the first column 202 may be used to separate the light and heavy components of the downstream MTO stream 201. According to one or more embodiments, the light components within the MTO stream 201 are vaporized and subsequently condensed, thereby forming a light component stream 203, while the heavy components are withdrawn to form a heavy component stream 204.

As mentioned above, the light component stream 203, comprising a mixture of tail gases, ethylene and ethane, from the first column 202 feeds the first dividing wall column 205 which purifies and separates ethylene 206 as a side stream. The mixture of tail gases is selected from the group consisting of CH₄, CO, N₂ and H₂. According to one of more embodiments, the dividing wall column removes the mixture of tail gases 208, recovers the ethane 207 and purifies and withdrawals the ethylene as an ethylene fraction 206 from a side stream of the post-fractionation section of the DWC 205. In one or more embodiments, the withdrawn ethylene fraction 206 is a polymer-grade ethylene or having a purity of at least 98%, or of at least 99.0%, 99.5%, 99.8% or 99.9%, in one or more embodiments.

As noted above, with reference to FIG. 2, the heavy component stream 204 from the first column 202 feeds the second column 210 where propylene is separated. According to one or more embodiments, the second column 210 vaporizes and condenses a propylene fraction 212. Furthermore, the heavy component stream 214 from the second column 210 feeds a third column 220 which separates the heavy component stream 214 into fractions. In one or more embodiments, the third column separates the heavy component stream to isolate a propane fraction 222, a mixed C4 fraction 224 and a C5+ fraction 226.

As mentioned above, with reference to FIG. 3, the heavy component stream 204 from the first column 202 may feed a second column 310. Thus, according to one or more embodiments, the second column is a dividing wall column. As mentioned above, when the second column 310 is a second DWC, propylene and propane are separated. In one or more embodiments, the second DWC 310 vaporizes and condenses propylene to thereby form a propylene fraction 312 and isolates propane 314, which is withdrawn as a side stream of the post-fractionation section. Furthermore, the second DWC 310 forms a heavy component stream 316 that feeds a third column 320. As mentioned above, the third column 320 may be used to separate the heavy C4/C5 component stream 316 into fractions. According to one or more embodiments, the heavy component stream 316 is separated into a mixed-C4 fraction 324 and a C5+ fraction 326.

As emphasized herein, another aspect of this disclosure is directed to a system for carrying out the purification of downstream MTO streams. Referring to FIG. 2, a generalized process flow diagram is shown to also represent a system for the purification of downstream MTO streams according to one or more embodiments. First, the downstream MTO stream 21 is fed to a first column 202 thereby separating the downstream MTO stream 21 into a light component stream 203 and a heavy components stream 204. The first column 202 may be fluidly connected to a first dividing wall column 205, thereby allowing the light component stream 203 from the first column to be received. According to one or more embodiments, the first dividing wall column 25 may be used to separate and produce an ethylene fraction 206.

The first column 202 may also be fluidly connected to a second column 210 thereby allowing the second column 210 to receive the heavy component stream 204 from the first column 202. According to one or more embodiments, the second column 210 separates the propylene fraction 212 and may be fluidly connected to a third column 220 thereby allowing the third column 220 to receive the heavy component stream 214 from second column 210. The third column 220 may be used to separate the heavy component stream 214 from the second column 210 into fractions. According to one or more embodiments, the third column separates the heavy component stream into propane 222, mixed-C4 224 and C5+ fractions 226.

Alternatively, and in accordance with one or more embodiments, the first column 202 may be fluidly connected to a second column such as a dividing wall column 310. According to one or more embodiments, the heavy component stream 204 is received by a second dividing wall column 310 where a propylene fraction 312 and propane 314 are separated. The second dividing wall column 310 may be fluidly connected to a third column 320, thereby allowing the heavy C4/C5 component stream 316 to be received by the third column 320. The third column 320 may be used to separate the heavy C4/C5 component stream 316 from the second dividing wall column 310 into fractions. According to one or more embodiments, the third column may be used to separate mixed-C4 324 and C5+ fractions 326.

While the scope of the system and method will be described with several embodiments, it is understood that one of ordinary skill in the relevant art will appreciate that many examples, variations and alterations to the system and methods described here are within the scope and spirit of the disclosure. Accordingly, the embodiments described are set forth without any loss of generality, and without imposing limitations, on the disclosure. Those of skill in the art understand that the scope includes all possible combinations and uses of particular features described in the specifications.

### Examples

The following examples are merely illustrative and should not be interpreted as limiting the scope of the present disclosure.

Using the Aspen Plus® software to model the separation of ethylene, propylene and butylene from an MTO process, three configurations are evaluated: 1) scheme with six conventional distillation columns; 2) scheme with three conventional distillation columns and one DWC; 3) scheme with two conventional distillation columns and two DWCs.

The feed stream conditions are shown in Table 1. The required purities for ethylene, propylene and butylene are 0.999, 0.996 and 0.99, respectively. As one of the aims of this work is to compare the three configurations in terms of energy, the total number of stages of each configuration was designed to be the same, so that there is uniformity during comparison.

TABLE 1

| Component | Units | Feed |
|---|---|---|
| Methane | (kg/h) | 352.60 |
| Ethane | (kg/h) | 335.54 |
| Propane | (kg/h) | 5572.20 |
| Nitrogen | (kg/h) | 17.89 |
| Hydrogen | (kg/h) | 332.53 |
| Carbon monoxide | (kg/h) | 90.12 |
| Ethylene | (kg/h) | 18599.00 |
| Propylene | (kg/h) | 10726.00 |
| C4+ | (kg/h) | 3400.15 |
| C5+ | (kg/h) | 658.18 |
| Total flow | (kg/h) | 40084.17 |
| Temperature | (° C.) | 20 |
| Pressure | (bar) | 15 |

### Comparative Example 1: Conventional System

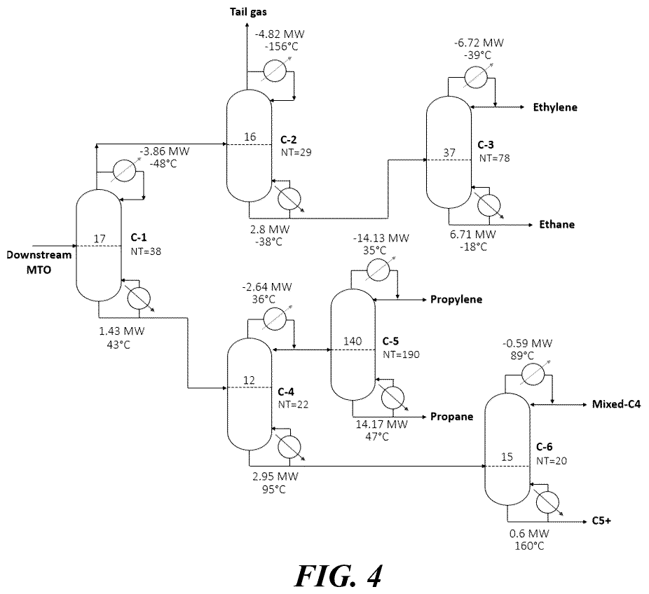

The UOP/HYDRO MTO process stream recovery section includes six distillation columns, as shown in FIG. 4. C-1 column separates ethane, ethylene and tail gases (top) from the propane, propylene and heavy olefin mixture (bottom). Demethanizing C-2 column separates tail gases CH₄, CO, N₂, H₂ from the ethane/ethylene mixture, which follows to the C-3 column, where polymer-grade ethylene is obtained. The base stream from C-1 follows to the C-4 column, which separates the propane/propylene mixture from heavy olefins. The propane and propylene obtained at the top of the column are directed to C-5, where polymer-grade propylene is obtained at the top. Heavy olefins are conducted to C-6, where butylene is obtained at the top, which can be used as fuel in MTO or in other processes.

The six distillation columns were simulated with the rigorous RadFrac model, from Aspen Plus and with details of streams given in Table 2. Partial condensers were used in C-1 and C-2 columns due to the large amount of light components in their feed and need for low condenser temperature. All other distillation columns in the process have total condensers.

TABLE 2

| | | Downstream | Tail | | | | | Mixed- | |
|---|---|---|---|---|---|---|---|---|---|
| Component | Unit | MTO | gas | Ethylene | Ethane | Propylene | Propane | C4 | C5+ |
| Methane | kg/h | 352.6 | 352.2 | 0.35 | 0 | 0 | 0 | 0 | 0 |
| Ethane | kg/h | 355.55 | 0 | 2.8 | 315.6 | 17.1 | 0 | 0 | 0 |
| Propane | kg/h | 5572.2 | 0 | 0 | 0.03 | 15.2 | 5546.9 | 10.01 | 0.00014 |
| Nitrogen | kg/h | 17.9 | 17.9 | 0 | | 0 | 0 | 0 | 0 |
| Hydrogen | kg/h | 332.5 | 332.5 | 0 | 0 | 0 | 0 | 0 | 0 |
| Carbon | kg/h | 90.12 | 90.12 | 0 | 0 | 0 | 0 | 0 | 0 |
| Monoxide | | | | | | | | | |
| Ethylene | kg/h | 18599 | 0 | 18577.4 | 18.6 | 3 | 0 | 0 | 0 |
| Propylene | kg/h | 10725.9 | 0 | 0 | 1.1 | 10679.1 | 41.8 | 3.9 | 0 |
| Butanes | kg/h | 3400.1 | 0 | 0 | 0 | 0 | 3.4 | 3383.4 | 13.3 |
| Petanes | kg/h | 658.2 | 0 | | 0 | 0 | 0 | 0 | 658.2 |
| Total flow | kg/h | 40084.2 | 792.8 | 18580.6 | 3350.3 | 10714.4 | 5592.1 | 3397.4 | 671.5 |
| Temperature | ° C. | 20 | 156.8 | −39.2 | −18.6 | 35.1 | 47.3 | 89.6 | 160.3 |
| Pressure | Bar | 15.1 | 15 | 15 | 15.6 | 15 | 16.2 | 15 | 15.1 |

Propylene recovery to desired specifications requires a 190-stage (C-5) column. Ethane/ethylene (C-3) separation requires 78 stages. Both have high specific energy consumption (0.36 MW/ton of ethylene and 1.32 MW/ton of propylene for C-3 and C-5, respectively). Columns that precede these two separations (C-2 and C-4) also have high energy requirement (around 2.9 MW each). Improvements that imply reductions in energy consumption for these four columns can economically impact the separation of streams from the MTO process, which encourages the application of techniques to intensify C-2, C-3, C-4 and C-5 distillation columns. The design specifications for the distillation columns are in Table 3 below.

TABLE 3

| Design | | |
|---|---|---|
| specifications | | |
| for distillation | | Variable |
| columns. | Specification | to adjust |
| C-1 | C₃H₆ Recovery = 99.99% (bottom) | Distillate rate |
| | C₂ Recovery = 99.99% (top) | Reboiler duty |
| C-2 | CH4 Recovery = 99.9% (top) | Distillate rate |
| C-3 | C2H4 Recovery = 99.9% (top) | Distillate rate |
| | C2H6 Recovery = 99.11% (bottom) | Reflux ratio |
| C-4 | C4 Recovery = 99.9% (bottom) | Reflux ratio |
| C-5 | C3H6 Recovery 99.6% (top) | Reflux ratio |
| C-7 | C3H8 Recovery = 99.5% (bottom) | Reboiler duty |
| | C3H6 Recovery = 99.7% (top) | Distillate rate |
| C-8 | C5 Recovery = 99% (bottom) | Reboiler duty |
| | C3H8 = 99.8% (top) | Distillate rate |
| DWC-2 | C₃H₆ Recovery = 99.8% (top) | Reflux ratio |

### Example 2: Intensified Process 1

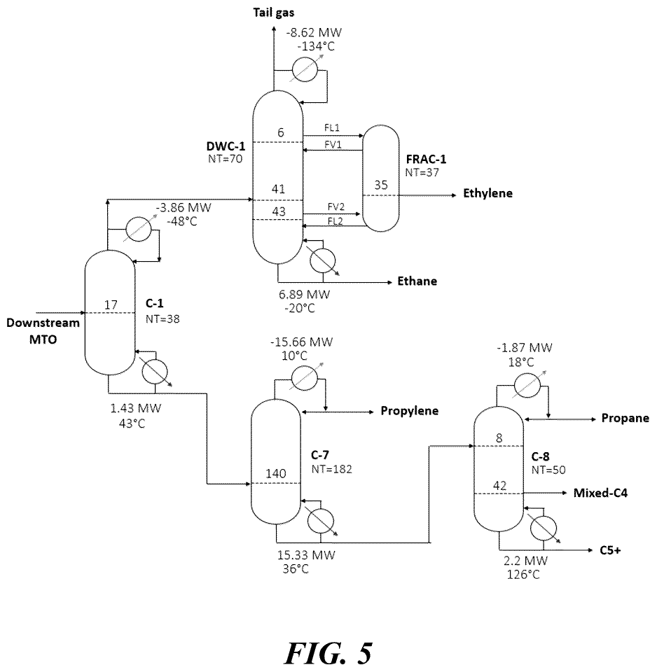

This example unifies C-2 and C-3 columns in DWC-1/FRAC-1 (205 in FIG. 2) and the three-column scheme (C-4, C-5 and C-6) is reduced to two columns, C-7 (210 in FIG. 2) and C-8 (with a side withdrawal) (220 in FIG. 3). This configuration is shown in FIG. 5.

The product removed at the top of the DWC-1 main column is composed of a mixture of tail gases (CH₄, CO, N₂, H₂), since they are lighter and have extreme volatilities compared to ethylene and ethane. The other key component with extreme volatility is ethane, which is removed at the bottom of the main column. Ethylene is removed in the side stream of the FRAC-1 post-splitter.

The three-column modification design (C-4, C-5 and C-6) reduced to two columns with side withdrawal (C-7 and C-8) used number of stages, feed stage and operating pressure according to Dimian and Bildea (2018). The side withdrawal stage, containing butylenes, was chosen according to the composition profile obtained in the Aspen Plus. Stage 42 was used to obtain the C₄ mixture at the required purity. At the top of C-7, propylene is obtained, while at the top and bottom of C-8, propane and C₅₊ are obtained, respectively.

The design of the proposed configuration was obtained by maintaining the total number of stages of the conventional scheme: C-2/C-3 becomes DWC-1/FRAC-1 (107 stages); C-4/C-5/C-6 becomes C-7/C-8 (232 stages). The number of stages of the rectifying section of the C-3 column was used as initial estimate to represent the post-fractionation section. The liquid and vapor withdrawal stages of the main column (FL1 and FV2) were selected according to the composition profile obtained: the section above the wall that divides the two sides of the DWC must contain only components A (tail gas) and B (ethylene), and the bottom section should only contain B and C (ethane).

To determine the DWC-1 reflux ratio (RR1) and FL1 and FV2 flows, sensitivity analyses were performed for these variables in order to minimize the thermal load of the reboiler, without compromising the desired purity of the product (ethylene). FL2 and FV1 flow rates are response variables referring to the liquid at the top and vapor at the bottom of the post-splitter (FRAC-1), respectively, which completely return to the main column (DWC-1).

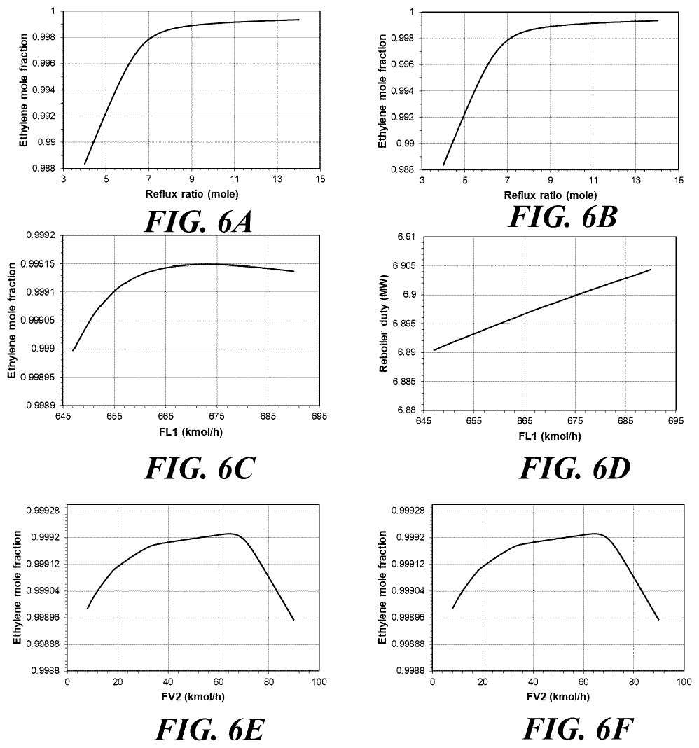

FIGS. 6A-6F present the results of the influence of the three manipulated variables (RR1, FL1 and FV2) on the thermal load of the DWC-1 distillation column reboiler and on ethylene purity. Increasing the reflux ratio implies an increase in ethylene purity up to the point where stability in the ethylene mole fraction in the distillate stream is achieved (FIG. 6A). The thermal load is also directly proportional to the reflux ratio (FIG. 6B). This result indicates that the choice of the optimal value for the reflux ratio (RR1=10.3) is the one in which the desired ethylene purity (99.9% mole) is achieved, since reflux ratio values above this will increase energy consumption without bringing relevant benefits to the purity of the product.

Regarding the variation in the liquid (FL1) and steam (FV2) flow that are removed from the main column and directed to the post-fractionation section (FRAC-1), the ethylene molar fraction reaches an optimum value (FIGS. 6C and 6E). In the case of FL1, the optimum value is above the desired purity; then, the chosen point (FL1=655 kmol/h) is the one where there is the lowest thermal load in the reboiler (FIG. 6D). For FV2, the chosen value is also the one in which the lowest thermal load is obtained (FV2=18 kmol/h) (FIG. 6F), when the desired purity is reached.

For DWC-1, the C₁/C₂/C₃ mixture was considered as the refrigerant used in the condenser, while for C-7/C-8 columns, the use of NH₃ was considered. Low pressure steam was adopted for the conventional column reboilers of this configuration, while for DWC-1, heating water at 20° C. was used to reach the required temperature of −20° C.

When compared to the conventional configuration in Example 1, this intensification scheme presented the following reduction in the thermal load: from 32.76 MW to 30.01 MW for condensers, and from 28.66 MW to 25.85 MW for reboilers.

The conventional configuration in Comparative Example 1 has the amount of refrigerant divided between two different temperatures, −156° C. and −39° C. The lowest temperature (−156° C.) has higher associated operating cost, which is different from the cost of the refrigerant used to reach the temperature of −39° C. (refrigerant with lower value compared to that of very low temperature). On the other hand, all the necessary refrigerant in DWC-1 is supplied at −134° C., which can increase operating costs (since all the necessary refrigerant is at a very low temperature). This also occurred with the change from C-4/C-5/C-6 (36˜89° C.) to C-7/C-8 (10˜18° C.).

### Example 3: Intensified Process 2

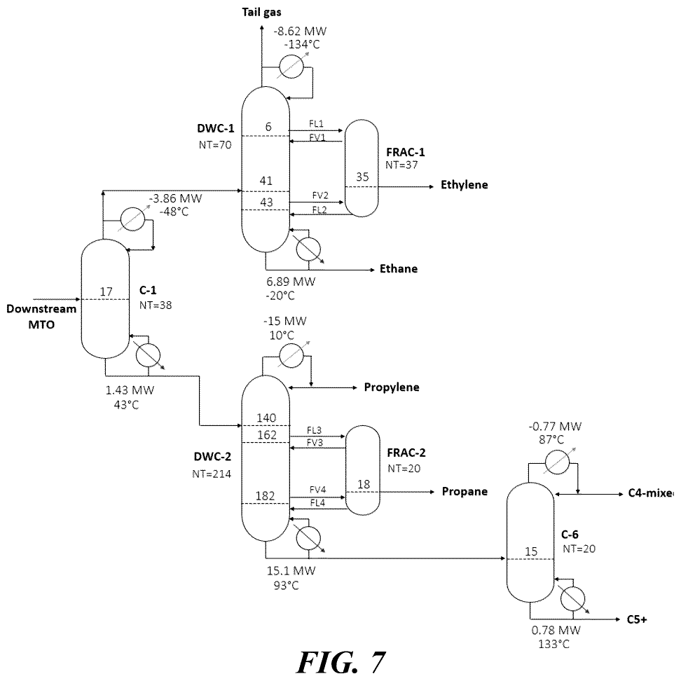

This example also uses DWC-1/FRAC-1 to separate ethane, ethylene and light gases. In addition, another modification is performed as compared to Comparative Example 1: C-4 and C-5 columns are replaced by a single DWC-2/FRAC-2 column, as shown in FIG. 7. Due to the good results previously obtained for DWC-1/FRAC-1, this item focuses on the second upgrade.

The design of DWC-2/FRAC-2 columns was conducted following the same approach previously described. The number of stages used in the DWC-2 column is 214. Generally, the industrial implementation of very tall columns is made possible by dividing the design hull into two physical hulls. In terms of simulation, this division does not impact the results.

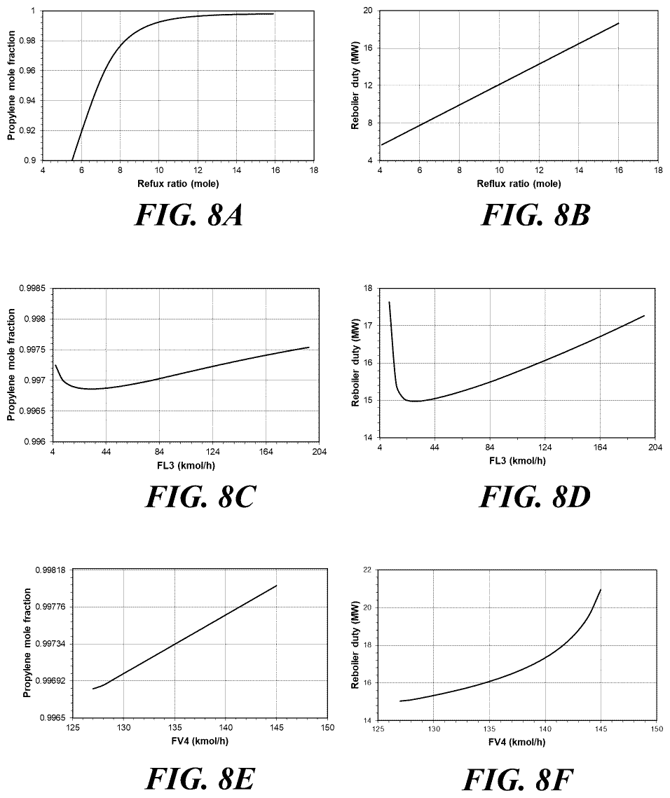

The results for the sensitivity analysis performed to determine the reflux ratio (RR2) and removals of liquid (FL3) and vapor (FV4) from the main column (DWC-2) are shown in FIGS. 8A-8F. The existence of parity between product composition, reflux ratio and reboiler thermal load was also observed (FIGS. 8A and 8B): the optimal value for the reflux ratio (RR2=11.8) is the one in which the desired propylene purity (99.6 mole %) is reached. According to FIGS. 8C and 8E, the propylene composition is above the desired one for all evaluated flow rates. Then, FL3 and FV4 flows were determined considering the minimum thermal load of the reboiler (FL3=41 kmol/h and FV4=128 kmol/h) (FIGS. 8D and 8F).

The condenser temperature is similar to that obtained in the C-7 column of the Intensified 1 configuration (10° C.). The DWC-2 column uses NH₃ as refrigerant, which can negatively influence operating costs; the conventional configuration uses cooling water. With the exception of DWC-1, which uses heating water, low pressure steam is used in all reboilers of this configuration.

Both dividing-wall columns, which replace four conventional columns, result in reduction in energy consumption from 26.63 MW to 21.99 MW for reboilers and from 28.31 MW to 23.62 MW for condensers.

### Economic Evaluation

### Energy

Table 4 presents the results in terms of energy for the three configurations evaluated (conventional, intensified 1 and intensified 2).

TABLE 4

| | Conventional | | Intensified 1 | | Intensified 2 | |
|---|---|---|---|---|---|---|
| | Reboiler | Condenser | Reboiler | Condenser | Reboiler | Condenser |
| Total energy | 28.66 | 32.76 | 25.85 | 30.01 | 24.2 | 28.25 |
| consumption | | | | | | |
| (MW) | | | | | | |
| Saving | — | — | 9.8% | 8.39% | 15.56% | 13.76% |

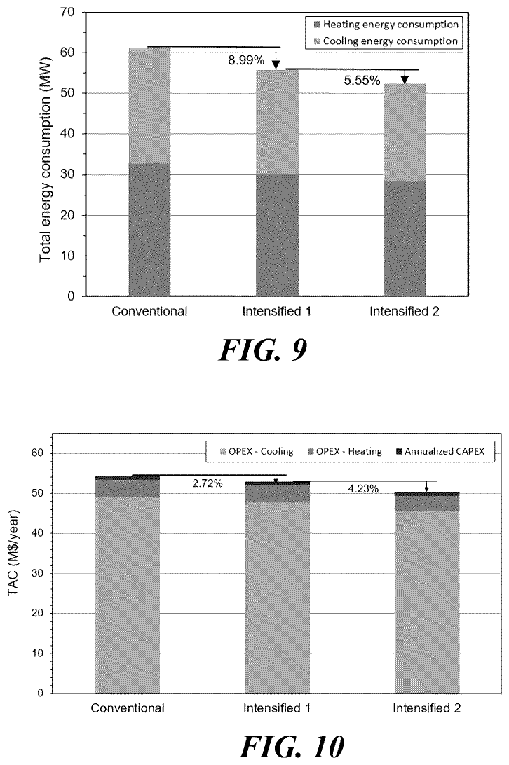

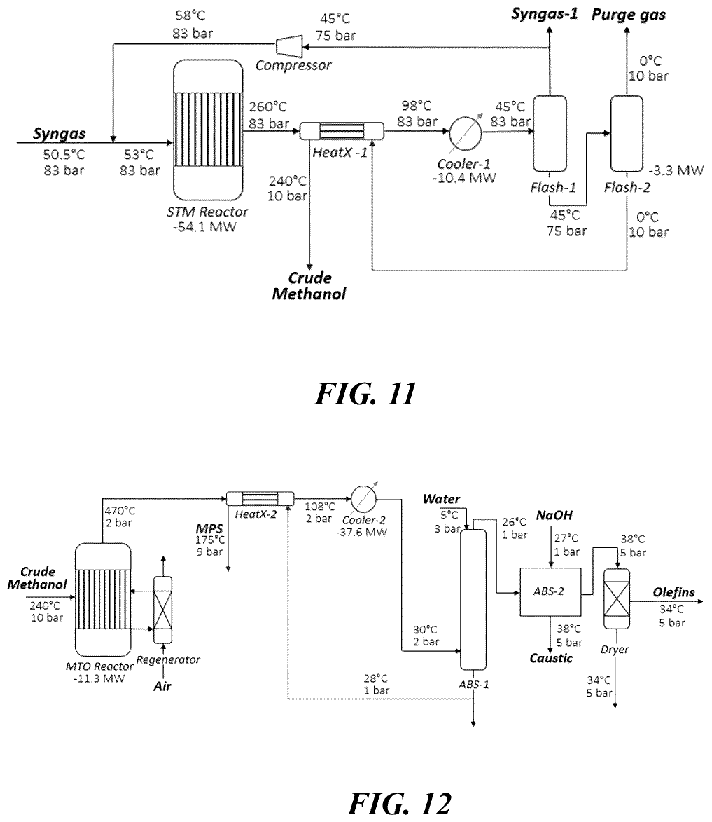

The configuration with two DWC (Intensified process 2) obtained the best results in terms of energy consumption reduction compared to the conventional scheme: 15.56% for the reboiler and 13.76% for the condenser. The configuration with only one DWC and three conventional columns also achieved improvements in relation to the conventional configuration, obtaining savings of 9.8% and 8.39% for the reboiler and condenser, respectively. These results are best evaluated graphically, as shown in FIG. 9. The Intensified 2 configuration presents the best energy performance: the total energy value associated with this configuration (reboiler+condenser) is 14.54% lower than the conventional sequence.

Evaluating DWC-1 separately and comparing it with results of C-1/C-2 columns, the reduction in energy consumption is 27.54% for the reboiler and 25.30% for the condenser. Comparing DWC-2 with C-4/C-5 columns, the reduction observed was 11.79% for the reboiler and 10.7% for the condenser.

Heating operating cost calculations were determined based on the price of the low pressure steam (7.78$/GJ), medium pressure steam (8.22$/GJ) and heating water costs (0.354$/GJ) (Luyben, 2011).

Cooling costs are more complex, as they take into account the very low temperatures at which some distillation columns in this study operate (down to −156° C.). Turton et al. (2009) evaluate the costs that the electrical consumption of a refrigeration system can generate. This system contains condenser, turbine, evaporator and compressor (75% efficiency). The analysis is carried out for temperatures down to −60° C., as temperatures below this value require cascade cooling.

Work by Luyben (2017) evaluates refrigeration costs for temperatures between −25° C. and −190° C. According to these examples, it is not only the electrical consumption generated in the system that is taken into account; refrigerant costs are also considered in the construction of the cooling system (with 1 or more stages), the purchase of refrigerant and the make-up used to replace losses. For this reason, the costs with refrigerants used in the present work were used according to the work by Luyben (2017), and are presented in Table 5.

TABLE 5

| | | |
|---|---|---|
| | | Refrigerant |
| T cold (° C.) | Refrigerant | cost ($/GJ) |

| | | | |
|---|---|---|---|
| −25° | C. | NH₃ | 18.5 |
| −50° | C. | C₃H₆ | 43.2 |
| −75° | C. | C₂/C₃ | 41.8 |
| −150° | C. | C₁/C₂/C₃ | 135 |
| −175° | C. | N₂/C₁/C₂/C₃ | 256 |

Since refrigerant and steam costs are different, it is more appropriate to evaluate results individually, as shown in Table 6.

TABLE 6

| | Conventional | Intensified 1 | Intensified 2 |
|---|---|---|---|
| Cooling ($/year) | 48,954,576.95 | 47,668,432.15 | 45,549,961.43 |
| Heating ($/year) | 4,431,085.77 | 4,355,730.39 | 3,714,148.41 |

Intensified configurations reduce heating costs by 1.7% (Intensified 1) and 16.17% (Intensified 2). Refrigerant costs can be annually reduced by 2.6% (Intensified 1) and 6.95% (Intensified 2). The Intensified 1 scheme achieved low reductions, in percentage terms, for heating costs. Despite the 25.3% energy reduction for the DWC-1 reboiler, the utility used in this reboiler has low associated costs (costs with low pressure steam, for example, are 25 times greater than with heating water), which does not bring relevant impacts for the reduction of operating costs. Furthermore, C-7 and C-8 columns also did not influence the reduction of operating costs, since the reduction in terms of energy was not significant. It is expected that the replacement of the three columns (C-4, C-5 and C-6) by two (C-7 and C-8) will imply improvements regarding capital costs. When the Intensified 2 scheme is analyzed, reductions in heating costs have greater impacts, as the optimized utility, with the energy reduction associated with the use of DWC-2, is low pressure steam, which has higher added value.

In percentage terms, the improvements obtained in costs with refrigerants are also low, but when the analysis is performed quantitatively (dollars/year), reductions with refrigerants represent more than double those obtained with steam, as a result of the high cost of the cooling system.

### Total Annual Cost

TAC is calculated through Eq.1 and was used as a comparison metric to evaluate the economic performance of the three configurations.

$$TAC ( \frac{\$}{year} ) = O P E Z + f * ( C A PEX ) \qquad ( 1 )$$

Eq.2 is used to calculate the annualization factor (f), which capitalizes the investment cost over the life of the equipment.

$$f = \frac{{i ( 1 + i )}^{n}}{{( 1 + i )}^{n} - 1} \qquad ( 2 )$$

where i represents the fractional interest rate per year and n the useful life of the equipment. Fixed values of 0.1 and 20 years were adopted for i and n, respectively (Christopher et al., 2017).

Operating costs (OPEX) considered refer to utilities (calculated in the previous topic). Operating time of 8000 hours per year was considered. Capital expenditures (CAPEX) for distillation columns are influenced by design data. Details on CAPEX-related calculations are shown in Table 7.

TABLE 7
Investment Capital ($/year) Description

| | |
|---|---|
| Column vessel | where |
| 17640D_{C}^{1.066}L_{C}^{0.802} | Dc = Column diameter (calculated through Aspen’s |
| | Column Internals); |
| | LC = Column height = NT trays with 2 ft spacing |
| | plus |
| | 20% extra length = 1.2*0.62*NT |
| Condenser/Reboiler | Where: |
| 7296 * A_{T}^{0.65} | ${A}_{T} = \frac{{Q}_{c / r}}{{U}_{c / r} dT}$ |
| | Qc/r = condenser/reboiler heat duty (kW), calculated |
| | Aspen Plus ® |
| | Uc/r = overall heat transfer coefficient for the |
| | $condenser / reboiler ( \frac{kW}{{m}^{2} K} )$ |

A penalty factor of 15% was adopted to offset the costs with the difficulty of constructing and installing the DWC (Li et al., 2020).

FIG. 10 presents the economic results for the three configurations. The sequence with two DWC had the lowest TAC ($50,214,705.63 per year). When compared to the conventional configuration, TAC reductions of the intensified schemes are 2.72% (Intensified 1) and 7.68% (Intensified 2). CAPEX does not present significant differences between the three configurations; the advantage of using a single hull is penalized through the construction costs of DWC. Therefore, the most relevant component of TAC is the utility costs (OPEX), which showed important reductions for intensified schemes.

Cooling costs account for approximately 90% of TAC in the three configurations (FIG. 10). Cryogenic distillation columns have high costs associated with the cooling system. Although recirculation of the fluid used as refrigerant is observed, high capital and operating cost (electricity), associated with the use of compressors is required, so that this fluid returns to the desired temperature. The influence of the cooling system, which is being taken into account in this work, causes TAC to present high values, which results in significant reductions in terms of dollars/year, for a 7.68% saving ($4,180,240.5 per year).

In distillation systems, CO₂ emissions are produced from furnaces and boilers, or due to the import of electricity. In this study, emissions resulting from the generation of steam in boilers, which is later used in reboilers, were considered.

CO₂ emissions are related to the amount of fuel burned, Q_{fuel} (KW), calculated from the energy balance of the steam generation process (Eq.3). Natural gas is considered as the fuel being burned for steam generation (Gadalla et al., 2006).

$${[ {CO}_{2} ]}_{e m i s s i o n} = \frac{{Q}_{f u e l}}{NHV} \frac{C \%}{100} α \qquad ( 3 )$$

where α is the ratio between molar masses of CO₂ and carbon (3.67);

$$NHV ( \frac{kJ}{kg} )$$

is the net calorific value of a fuel with carbon content of C % (dimensionless); and C %=75.4 for natural gas. The amount of fuel required (Q_{fuel}) is calculated according to Eq.4 (Gadalla et al., 2006).

$${Q}_{fuel} = \frac{{Q}_{p r o c e s s}}{{λ}_{p r o c e s s}} ( {h}_{p r o c e s s} - {h}_{f}^{H 2 O} ) \frac{{T}_{FTB} - {T}_{0}}{{T}_{FTB} - {T}_{s tack}} \qquad ( 4 )$$

where λ_{process} (kJ/kg) and h_{process} (kJ/kg) are the latent heat and enthalpy of the steam delivered to the process, respectively; T_{FTB} is the boiler flue gas flame temperature (1800° C.); To is the ambient temperature; T_{stack} is the temperature of generated combustion gases (160° C.) (Gadalla et al., 2006); and Q_{process} is the thermal load required by the system, obtained by Aspen Plus.

The estimate of CO₂ emissions for each evaluated configuration is presented in Table 8. The two intensification proposals present better carbon footprint in relation to the conventional configuration. The environmental impact can be reduced by up to 14.19% by replacing four conventional columns with two dividing-wall columns (Intensified 2). So, while intensification of the MTO downstream separation process brings reductions in CO₂ emissions, profitability can also be increased with reduction in total annual costs, making this type of configuration an attractive option for this process.

TABLE 8

| | Conventional | Intensified 1 | Intensified 2 |
|---|---|---|---|
| ${CO}_{2} emission ( \frac{{t CO}_{2}}{day} )$ | 79.82 | 70.68 | 68.49 |
| Saving CO₂ (%) | — | 11.44% | 14.19% |

### CONCLUSIONS

Two intensified configurations were proposed for the MTO downstream separation process and compared with the conventional sequence with six distillation columns. The comparison was carried out from the energy, economic and environmental perspective (CO₂ emissions).

The use of two DWC (Intensified 2) was the sequence that presented the best results. Costs with utilities used in the reboiler had reductions of up to 16.17%. Refrigerant costs demanded a more accurate calculation, since very low temperatures are used in condensers. These calculations included cooling system construction costs, electricity consumption and make-up of refrigerant losses. Despite the high expenses with refrigerants, the use of dividing-wall columns resulted in savings of up to 6.95% over the conventional configuration. Regarding TAC, the Intensified 2 sequence showed reductions of up to 7.68%. It was also possible to mitigate the environmental impact: improvements of up to 14.19% were obtained, which reduced daily CO₂ emissions by 11.33 tons.

Therefore, the intensification of the MTO downstream separation process using DWC is a promising technology to reduce energy consumption, capital and CO₂ emissions.

### Example 4

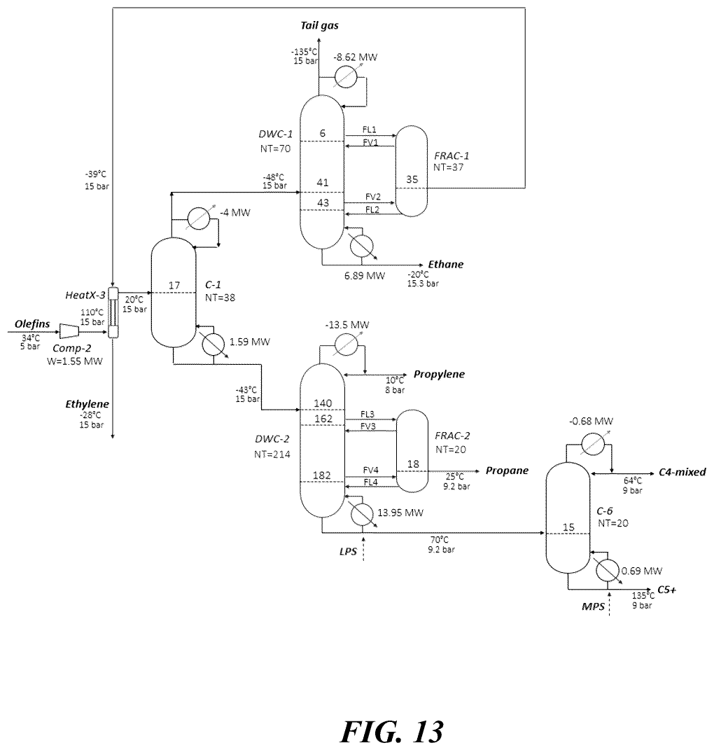

A design proposal that focuses on recovering and reusing heat generated during the synthesis gas to methanol (STM) and MTO reactions, while integrating the principle of process intensification in the olefin separation system is evaluated. In the section dedicated to converting synthesis gas into methanol, there is a crucial step of cooling the methanol produced in the reactor from 260° C. down to 45° C., where the separation of unreacted synthesis gas and methanol occurs (Flash-1). A second flash unit (Flash-2), operating at a lower temperature of 0° C., is employed for the removal of waste gases. Following the separation in these flash units, the resultant stream may be reheated to a temperature of 240° C., a prerequisite for its entry into the MTO reactor. The adopted strategy for achieving this involves the innovative reuse of heat from the output stream of the STM reactor, as illustrated in FIG. 13.

A heat exchanger, termed HeatX-1, is introduced to facilitate heat transfer between the cold stream emerging from the flash unit (at 0° C.) and the hot stream exiting the reactor (at 260° C.). In the MTO reactor, the reactions produce a significant amount of water and are highly exothermic, resulting in a reactor exit stream temperature of 470° C. This stream needs to be cooled before it can proceed to the purification stage.

The primary goal of the energy integration in the reaction section of the (MTO) process is the strategic reuse of a portion of the water extracted in the absorber (ABS-1), a byproduct of the reactions in the olefin production from methanol. This step involves generating steam in a unit named ‘HeatX-2’, using the heat from the MTO reactor's output stream. Concurrently, this unit aids in dissipating some of the heat from the MTO reactor's output, as shown in FIG. 12. The steam generated from the heat released in the conversion of methanol to olefins can then be beneficially utilized in the distillation columns of the olefin separation section.

The steam generated from the heat released in the conversion of methanol to olefins can then be beneficially utilized in the distillation columns of the olefin separation section. The separation section has been restructured as shown in FIG. 13, where two dividing-wall distillation columns (DWC) were used alongside two conventional columns. The design of the DWC-1/FRAC-1 and DWC-2/FRAC-2 columns followed the same approach as previously described.

Medium-pressure and low-pressure vapors, critical for the operation of DWC-2 and C-6, are produced in the MTO reaction section. These vapors are initially directed to the C-6 column, which requires medium-pressure steam, and then to the DWC-2 column, designed to operate with lower pressure steam. It's important to note that the olefin-rich gas from the drying process needs to be compressed to achieve the pressure levels required for the distillation columns. This compression increases the temperature of the gas, necessitating its cooling before it is fed into the C-1 column, which operates at cryogenic temperatures (−48° C. at the top and −43° C. at the bottom). To optimize utility consumption during this cooling stage, the design includes utilizing the ethylene stream exiting FRAC-1. With its temperature of −39° C., it effectively cools the stream feeding into C-1.

### Energy

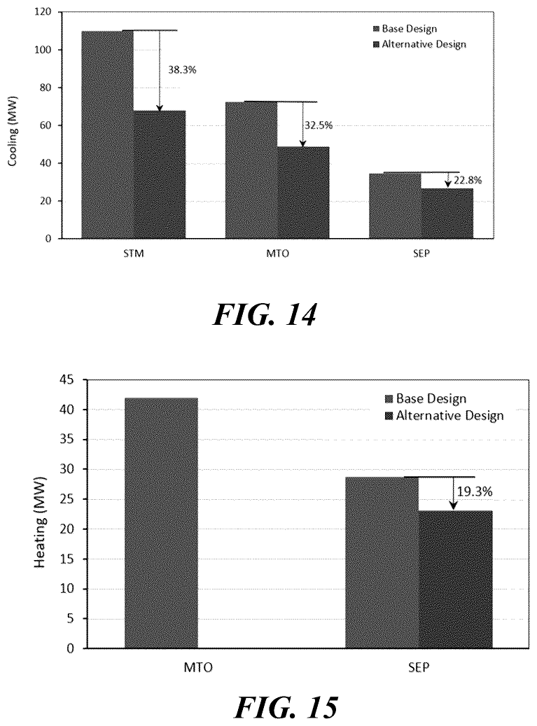

FIG. 14 provides a comparative analysis of the energy required for heat dissipation across three simulated stages, contrasting the base design with the alternative design. In the synthesis gas-to-methanol conversion section, the traditional setup requires −109.8 MW for heat removal, whereas the alternative design reduces this to −67.8 MW, marking a 38.3% reduction. In the methanol-to-olefins conversion stage, the alternative design also shows significant energy efficiency improvements. The cooling demand drops from −72.4 MW in the base design to −48.9 MW in the alternative design, a reduction of 32.5%. In the separation section, the alternative design achieves a 22.8% improvement in energy efficiency for heat dissipation, lowering the energy demand from −34.7 MW in the base design to −26.8 MW in the alternative design. Cumulatively, these improvements lead to a 33.8% reduction in heat dissipation for the entire process when adopting the alternative design.

Regarding the energy used in heating processes, as shown in FIG. 15, it is important to note that the STM section operates without any energy demand in this scenario, thus it is not included in the graph. In the MTO section, the base design requires 42 MW of energy to preheat the stream for the reactor. However, with the implementation of energy integration, the need for external energy supply in this section is completely eliminated, resulting in no energy demand.

In the olefin separation stage of the base design scenario, reboilers account for an energy demand of 28.66 MW. The integration of two dividing-wall distillation columns in the alternative design reduces this figure to 23.12 MW, indicating a 22.8% decrease in energy consumption. Consequently, the overall energy supply demand (heating) for the entire process experiences a significant drop from 70.66 MW in the base design to 23.2 MW in the alternative design, reflecting a substantial 67.3% reduction.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims. In the claims, means-plus-function clauses are intended to cover the structures described herein as performing the recited function and not only structural equivalents, but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures. It is the express intention of the applicant not to invoke 35 U.S.C. § 112 (f) for any limitations of any of the claims herein, except for those in which the claim expressly uses the words ‘means for’ together with an associated function.

What is claimed is:

1. A process of purifying downstream methanol-to-olefin streams comprising:
separating a downstream methanol-to-olefin stream comprising one or more light and heavy components in a first column into a light component stream and a heavy component stream;
purifying a light component stream with a first dividing wall distillation column thereby producing an ethylene fraction; and
distilling the heavy component stream thereby forming a propylene fraction.

2. The process of claim 1, wherein the downstream methanol-to-olefin stream comprises at least one selected from the group consisting of methane, ethane, propane, nitrogen, hydrogen, carbon monoxide, ethylene, propylene, mixed-C4, mixed-C5 and mixtures thereof.

3. The process of claim 1, wherein the separating further comprises:
vaporizing and subsequently condensing the one or more light components thereby forming the light component stream; and
withdrawing the heavy components thereby forming the heavy component stream.

4. The process of claim 1, wherein the first dividing wall distillation column comprises a single reboiler and condenser, and a partition wall therein, dividing the column into a post-fractionation section and a main column.

5. The process of claim 1, wherein the ethylene fraction is withdrawn as a side stream from the post-fractionation section.

6. The process of claim 1, wherein the purifying step further comprises:
removing a mixture of tail gases; and
recovering ethane.

7. The process of claim 6, wherein the mixture of tail gases is selected from the group consisting of CH₄, CO, N₂ and H₂.

8. The process of claim 1, wherein the ethylene fraction is polymer-grade ethylene, or having a purity of at least 98%.

9. The process of claim 1, wherein the distilling step further comprises:
vaporizing and condensing propylene in a second column, thereby isolating the propylene fraction.

10. The process of claim 9, wherein the second column is a dividing wall distillation column.

11. The process of claim 10, further comprising withdrawing propane as a side stream.

12. The process of claim 10, further comprising, withdrawing a heavy C4/C5 component stream and separating it in a third column into a C4 fraction and a C5+ fraction.

13. The process of claim 9, further comprising:
vaporizing and condensing propane in a third column thereby isolating propane;
withdrawing a C4 fraction as a side stream from the third column; and
isolating a C5+ fraction.

14. A system for purifying downstream methanol-to-olefin streams, comprising:
a first column for separating the downstream methanol-to-olefin stream a light component stream and a heavy component stream;
a first dividing wall distillation column fluidly connected to the first column, receiving the light component stream and producing an ethylene fraction therefrom; and
a second column fluidly connected to and receiving the heavy component stream from the first column, the second column configured to isolate a propylene fraction from the heavy component stream.

15. The system of claim 14, further comprising: a third column fluidly connected to and receiving the heavy component stream from the second column, the third column separating the heavy component stream into a propane fraction, a C4 fraction, and a C5+ fraction.

16. The system of claim 14, wherein the second column is a second dividing wall distillation column configured to isolate the propylene fraction and a propane fraction from the heavy component stream and withdraw a heavy C4/C5 component stream.

17. The system of claim 16, further comprising: a third column fluidly connected to and receiving the heavy C4/C5 component stream from the second column, the third column separating the heavy C4/C5 component stream into a C4 fraction and a C5+ fraction.
